(19) United States
(12) Patent Application Publication
(10) Pub. No.: US 2003/0180776 A1
(43) Pub. Date: Sep. 25, 2003

(54) Detection by sliding template amplification

(76) Inventors: Ming Wu, Castro Valley, CA (US); Edwin F. Ullman, Atherton, CA (US)

Correspondence Address: HANA VERNY, PETERS, VERNY, JONES & SCHMITT LLP, SUITE 6, 385 SHERMAN AVENUE, PALO ALTO, CA, 94306, US

(21) Appl. No.: 10/371,600

(22) Filed: Feb. 20, 2003

Related U.S. Application Data
(60) Provisional application No. 60/359,223, filed on Feb. 21, 2002.
(60) Provisional application No. 60/379,360, filed on May 8, 2002.

(51) Int. Cl. C12Q 1/68; C12P 19/34

(52) U.S. Cl. 435/6; 435/91.2

(57) ABSTRACT

Methods and compositions are provided for amplifying tandem repeats, particularly for detecting binding events, using a nucleic acid reagent comprising two features, one having a tandem repeat region and the other having an extendable 3′ terminus. When the two oligonucleotides are hybridized in the presence of a DNA polymerase and NTPs for replicating the tandem repeat region, repetitive extension of the extendable 3′ terminus along the tandem repeat region is obtained. By having labeled NTPs, the amplification can be detected as indicative of the binding event. Kits are provided for performing the method.

### CROSS-REFERENCE TO RELATED APPLICATIONS

This application is based on provisional applications serial Nos.60/359,223 and 379,360, filed respectively Feb. 20, 2002 and May 8, 2002.

### BACKGROUND OF THE INVENTION

### FIELD OF THE INVENTION

The invention concerns methods of detection of an analyte using repetitive extension along a tandem repeat.

### BACKGROUND INFORMATION

Advances in the biological sciences continue to accelerate, as the functions of the cell, including the genome, proteome, proliferation and differentiation are elucidated. With the sequencing of prokaryotic and eukaryotic, non-vertebrate and vertebrate genomes, the opportunity now exists to compare the evolutionary developmental processes, the differences between species, the genomic individuality among members of a species, and how the differences have influenced the phenotype. With the advent of proteomics, involving transcriptomes, translatomes, secretomes, and interactomes, the biological sciences are at the inception of a revolution of our understanding of the processes of biological development, genetic and infectious diseases, and manipulating biological entities.

As part of the biological advance, there is a need for sensitive methodologies that permit detection of low level events, where the amount of the material of interest is a very small proportion of the total of like material present. With nucleic acid detection, there are always numerous components that can compete to varying degrees with the analyte for the detecting entity. Since there is a concentration effect, with the target sequence present in small amount, substantial interference can occur with sequences having mismatches. One can reduce mismatched sequence binding by employing conditions of high stringency, but this makes the complex formation of the target sequence and its homologous sequence a very low level event. Toward this end, labels that can be readily amplified are of great interest. The stronger the signal and the lower the interference, the fewer false positives and negatives will occur and the more robust will be the determination. There has been a continuing effort in identifying new labels that provide the desired properties in a variety of contexts.

### RELEVANT LITERATURE

Tandem repeat expansion is described by Nakayabu, et al., Nucleic Acids Res. 1998, 26, 1980-4; Lyons-Darden, et al., J. Biol. Chem. 1999, 274, 25975-8; Oshima, et al., J. Biol. Chem. 1997, 272, 16798-806; Kunkel, et al., Proc. Natl. Acad. Sci. USA 1994, 91, 6830-4; Madsen, et al., Proc. Natl. Acad. Sci. USA 1993, 90, 7671-5; Ulyanov, et al., Struct., Motion, Interact. Expression Biol. Macromol. Proc. Conversation Discip. Biomol. Stereodyn, 10^{th} (1998) Meeting, Date 1997, 1, 75-88. Publisher: Adenine Press, Schenectady, N.Y.; Viguera, et al., EMBO 2001, 20, 2587-95; da Silva and Reha-Krantz, J. Biol. Chem. 2000, 275, 31528-35; and Gacy and McMurray, Biochemistry 1998, 37, 9426-34. Detection of short tandem repeats with arrays is described by Radtkey, et al., Nucleic Acids Res. 2000, 28, el7, ii-vi. U.S. patents of interest concerning immunoassays, with nucleic acid analytes and other analytes Pat. Nos. 4,785,080; 4,921,788; 4,937,188; and 5,656,731.

### SUMMARY OF THE INVENTION

The present invention relates to reagents providing label signal augmentation. The reagent has at least two functional components or features, where the two components comprise hybridizable nucleic acid sequences for complexing of the two components, one feature being a tandem repetitive sequence as a template, and the other feature being a 3′ extendable terminus. The reagents are combined with a polymerase and dNTP(s) to form a reagent system. When the components are complexed the 3′ extendable terminus is extended along the tandem repetitive sequence with slippage and further extension providing a replication copy expanded as compared to the repetitive sequence. Various methods can be used to detect the replication copy, hereinafter called the amplicon or amplified complementary tandem repeat sequence, including labeling the repetitive template, labeling NTPs, detecting the molecular weight of the amplicon, or other technique. The reagents and method can be used for determining any binding event, including identifying nucleic acid sequences, ligands and receptors.

### BRIEF DESCRIPTION OF THE DRAWINGS

FIG. 1 is an electropherogram indicating the sequences employed and the concentrations of the sequences;

FIG. 2 is a diagram of the proposed mechanism of the subject invention.

FIG. 3 is an electropherogram of a time course of the repetitive extension according to the subject invention.

FIG. 4 is an electropherogram as to the effect of varying the NTPs on repetitive extension.

FIG. 5 is an electropherogram of the effect of excess of a template sequence on the appearance of the template in the gel in a denaturing and non-denaturing gel.

FIG. 6 is a cartoon using a stem/loop probe to identify a target sequence with repetitive extension of the probe according to the subject invention.

FIG. 7 is an electropherogram of the products using a stem/loop probe in solution to identify a target sequence with repetitive extension according to the subject invention.

FIGS. 8a and 8b compare the effect of using polyA or polyT as the template for repetitive extension according to this invention.

### DETAILED DESCRIPTION OF THE INVENTION

Methods and compositions are provided for detection of an analyte by repetitive extension of the 3′-terminus of a nucleic acid along a template tandem repeat. The extended nucleic acid may be used for detecting a moiety, particularly involved in a binding event employing a reagent comprising a reagent composition. The reagent composition used in the method has two components or features that provide for an extendable label for detection. DNA polymerase and at least one nucleotide triphosphate (NTP) are included in the system. Depending on the methodology and protocol, as well as other entities to which one or both components may be bound, the individual components will vary as to composition and purpose.

The components of the reagent may be separate molecules or a single molecule. For example, a single molecule may be used where one component comprising a 3′ extendable terminus, the extendable oligonucleotide component, is joined by a linker to the other component, comprising a template member or tandem repeat template having a sequence positioned 3′ of a tandem repeat that is complementary with the 3′ extendable terminus component. Alternatively, these two components may be two molecules that are not joined by a linker.

In its simplest form, the subject reagent may be used to provide large tandem repeat molecules having at least about 100 repeats, more usually at least about 200 repeats and may provide repeats of a 1000 or more, depending on the size of the repeat unit, there being more repeats with smaller sized repeat units. The number of repeats resulting from the replication of the tandem repeat template may be 2 times, usually at least about 5 times and frequently 20 times or more than the number of repeats of the tandem repeat template.

Frequently, a component of the reagent may be used as a label. In this situation, wherever it is desired to detect a molecule to which a component of the reagent is bound, one may induce repetitive extension of the tandem repeat. As indicated, the label may be a single molecule with both of the reagent features or two different molecules, with one molecule having one feature and the other molecule having the other feature. Events can be determined, such as a different situs for the moiety, change in conformation, etc. The reagent can also be used for the detection of the presence of a moiety, where the moiety can exist in two different states, particularly binding events, where the moiety is involved in binding to a second moiety or is unbound. For the most part, binding events will be of interest.

Such binding events include the detection of nucleic acid sequences where complementary sequences hybridize, the binding of a ligand and receptor where a reagent component can serve as a label, or any other situation where an analyte is to be determined and amplification of a signal is desirable. The subject invention may be divided into two parts, one part where the target is a nucleic acid and the protocol involves the hybridization of one of the components to the target. The other part involves the use of a component of the reagent as a label for amplifying signal, where the target will usually be other than a nucleic acid, but need not be, and will usually involve a specific recognition reagent other than a nucleic acid. The application to target nucleic acids will be considered first.

The two reagent components are oligonucleotides, which in the presence of a polymerase and at least one NTP, complex with each other and the extendable oligonucleotide component is repetitively extended as a result of slipping along the tandem repeat of the template member. The salient characteristics of the two components are, respectively, a 3′-extendable terminus and, hybridized to it, a template member having tandem repeats at or 5′ of the site of hybridization. The oligonucleotides will usually be comprised of natural bases, such as dexoy- and ribonucleotides and their derivatives, such as T (or U), A, G and C, but unnatural bases may be used at sites where they do not interfere with the polymerase catalyzed repetitive extension. Examples of unnatural bases include without limitation 2′-methoxynucleotides, deazaadenosine, inosine, 5-bromouridine, 5-bromocytosine, 7-deazaguanasine, 8-bromoadenosine, 5-aminoallyluridine, 5-methylcytidine phosphates and phosphonate analogs of nucleotides, and the like, nucleotides having a detectable label and nucleotide mimetics such as, for example, phosphorothioates; protein nucleic acids (PNA); 2′-modified nucleosides, and the like. The oligonucleotide sequences may also be a combination of natural and unnatural nucleotides. The requirement is that the 3′ terminal nucleotide must be capable of extension.

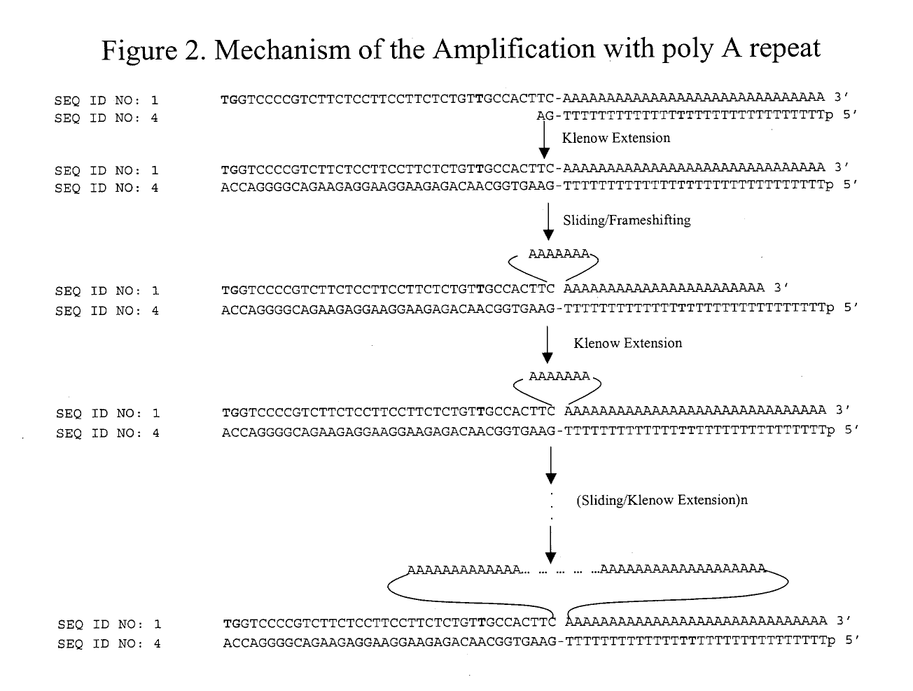

A key element of the subject method is diagrammed in FIG. 2. SEQ ID NOS: 1 and 4 are brought together whereby SEQ ID NO: 4, the template member, hybridizes to the extendable oligonucleotide component, SEQ ID NO: 1, which has a free 3′-end. The polyT tandem repeat binds to the polyA of SEQ ID NO: 1, with the dinucleotide at the 3′ terminus of SEQ ID NO:4 hybridized to the dinucleotide of SEQ ID NO: 1 immediately preceding the polyA segment. The Klenow fragment of DNA polymerase and ATP are added. Although not required by the method of this invention, in this illustration the 3′ end of the template member is extended along the extendable oligonucleotide. The 3′-end of the poly A segment slips one or more bases along the tandem repeat with dissociation of a like number of base pairs elsewhere in the duplex and is then extended, so that one or more polyA loops are formed. This process of slipping and extension is repeated as the polyA loop(s) become(s) enlarged.

Either component of the reagent may bind to or be part of a target nucleic acid. Often the extendable oligonucleotide component will comprise part of the target and be capable of binding to and repetitively extending along the tandem repeat of the template member. Alternatively the extendable oligonucleotide component can hybridize with, or is otherwise attached to, the target in a manner that provides that its 3′-end remains capable of being extended along the template member. In this case the 3′-end of the extendable oligonucleotide will comprise 2 or more, usually 6 or more, bases that are not hybridized to the target. On some occasions the target may contain a tandem repeat sequence that will allow it to serve as the template member. An example of such a target is mRNA that has a 3′-polyA tail. In such cases the target is able to hybridize with the extendable oligonucleotide component in a manner that will allow repetitive extension of its 3′-end. Alternatively the template member can hybridize with or is otherwise attached to the target in a manner that provides that the sequence comprising the tandem repeat template remains unhybridized to the target and free to hybridize to the free 3′-proximal region of the extendable oligonucleotide component.

The use of the subject reagent for determining binding events will involve distinguishing between bound and unbound target. The distinction can be as a result of being bound to a surface, where unbound reagent may be removed, being in an active state as distinguished from an inactive state, e.g. steric hindrance, particularly as a result of a binding event, or change in conformation, where binding is inhibited in the inactive state, sequestered in an environment where the target is not available, etc.

Various combinations of reagents may be employed of lesser or greater convenience. One may have an intermediate binding member comprising an oligonucleotide, where the intermediate binding member serves to bind specifically to the target and has a recognition region for one of the reagent components. In this situation the polarity of the various oligonucleotides can be varied as desired, so long as the reagent component has a 3′ extendable terminus and a tandem repeat region replicatable from the 3′ extendable terminus. By combining the sample with the intermediate binding member under hybridizing conditions, where the resulting complex is sequestered, one may then add the reagent components and the remaining components of the reagent system. The recognition region for a reagent component may be an oligonucleotide sequence capable of hybridization to the reagent component or a ligand or receptor that binds to a corresponding receptor or ligand comprising the reagent component. An alternative for nucleic acid detection is where one component, the template member, will have three regions, a first region capable of hybridizing to the target, a second region that hybridizes to the 3′ proximal region of the second component, and a third region 5′ of the hybridization site comprised of a tandem repeat region. The second extendable oligonucleotide component need have only one region, a hybridizing region for hybridizing to the first component and terminating in an extendable 3′-end. Alternatively the extendable oligonucleotide component can have a first region capable of hybridizing to the target and a second region comprising an extendable 3′-end that hybridizes to a site on the template member. In this case the template member need only have a tandem repeat region 5′ of the hybridization site.

Depending upon the role the components in the assay are to play, the number of nucleotides will vary greatly. In the first instance, the nucleic acid target will be considered. The nucleic acid target can be DNA or RNA and will vary widely in length from as few as 10 bases up to millions of bases or an entire chromosome. Usually the target will be mRNA, cDNA or a nucleic acid amplicon, which may vary in length from 50 to 10,000 or more bases.

The extendable oligonucleotide component needs only a 3′-end region for binding to another nucleic acid with the desired level of specificity. When not part of the same molecule comprising the template member, the extendable oligonucleotide component will be an oligonucleotide of at least about 8, more usually at least about 12, nucleotides, and, when not part of the target, generally not more than about 500, more generally not more than about 100, usually not more than about 50 nucleotides. The maximum number of bases is determined more by economics and convenience, than by operability, so long as one obtains the desired degree of specificity and operability. Depending upon whether the reagent comprising the label is comprised of one or two molecules, for two molecules the extendable component will usually have from one to two regions or segments. Where the reagent is serving as a label and bound to a detection entity, normally other than a nucleic acid, then the segment will have a recognition sequence for the second component and an extendable 3′ end, where the recognition sequence may be complementary to the tandem repeat sequence of the template sequence

As previously described, where one of the components is used in conjunction with detection of a nucleic acid sequence, the component may have two regions, where the first region will be complementary to the target sequence and the second region will be complementary to the second component of the reagent. Therefore this component serves as a bridge between the target and second component, where one region identifies the target and the second region serves as the 3′ extendable terminus or template tandem repeat required to initiate repetitive extension of the 3′ extendable terminus. The region complementary to the target will usually have at least about 8 nucleotides, more usually at least about 15 nucleotides. It should be understood that the two regions may be linked by a bond or any other linker of any length that does not interfere with the functions of the components. As a matter of convenience the linker will usually comprise a chain of fewer than 500 atoms, usually fewer than 100, and may consist of other than nucleotides such as abasic sites, polyethers, polypeptides, etc, where such linkage may serve simply as a connecting unit or may have value in providing for cleavage, enhancing detection, etc.

When the reagent has two different molecules, the template member serves to bind to the extendable oligonucleotide component and provide a tandem repeat template for the extension of its 3′ extendable terminus. The template member will have a recognition region for binding to the extendable component and a tandem repeat region. These regions may be the same or may be separate or overlapping regions where the recognition region is located 3′ of the 5′ end of the tandem repeat. The recognition region will usually have at least about 4, more usually at least 6, and preferably at least 12 nucleotides and generally not more than about 50, more generally, not more than about 30 nucleotides. Preferably the recognition region will not be separated from the tandem repeat but when separated will be separated by any convenient polynucleotide sequence that can serve as a polymerase template. The template region will be comprised of short repeating sequences, where the repeating sequence will generally be from about 1-6, more usually 1-4, nucleotides, and preferably 1-2 nucleotides. Depending on the size of the repeat, there will be at least about 2 repeats, more usually at least about 8 repeats, generally not more than about 100 repeats, usually not more than about 50 repeats, and frequently at least about 20 repeats. Usually, the longer the repeat sequence, the fewer the number of repeats. With repeats having from about 1-3 nucleotides, the number of nucleotides in the tandem repeat region will generally be at least about 8, while with repeats having from about 4-6 nucleotides, the number of nucleotides in the tandem repeat region will usually be at least about 10. Usually the template member will be at least about 8 nucleotides, usually at least about 12 nucleotides, more usually at least about 20 nucleotides, desirably at least about 30 nucleotides and may be 60 nucleotides or more, generally not more than about 200 nucleotides.

When the reagent is a single molecule, the molecule will usually be capable of having a stem/loop structure, with a first arm being the template member attached covalently to the other arm being the 3′ extendable oligonucleotide component and hybridizable to the first arm. Conveniently the 3′ end of the template member will be attached to the 5′ end of the extendable oligonucleotide component through a linker that comprises a loop when the two arms are hybridized to each other. However other sites of attachment may be used. A bond or any convenient chain of atoms, usually consisting of fewer than about 100 atoms, preferably 1-60 atoms, and more frequently 1-30 atoms, can be used as the linker. For example, the first arm and loop could be polyT, connected at its 3′ end to the other arm which would have a 3′ terminal polyA sequence.

Tandem repeat sequences may be illustrated by (A)ₙ, (T)ₙ, (C)ₙ, (G)ₙ, (ATT)ₙ, (AAC)ₙ, (AAG)ₙ, (TTC)ₙ, (TTG)ₙ, (GTTT)ₙ, (CAAA)ₙ, (AAATC)ₙ, (AAATG)ₙ, (TATTG)ₙ, etc. where n is the number of repeats. Desirably, the tandem repeat sequence will not be palindromic to avoid self-hybridization. Preferably, the repeats will be comprised primarily of As and/or Ts, desirably solely of As and/or Ts.

For the most part, the nucleotide triphosphates (NTPs) that are employed will be the naturally occurring deoxynucleotides, dATP, dTTP, dCTP and dGTP or analogs thereof. In particular analogs having a detectable label such as a fluorescent or chemiluminescent label or a hapten to which a labeled binding agent can bind, such as biotin will frequently be used. The labeled NTPs provide detection of the extension reaction. NTP analogs that have lower binding to their complementary bases than the naturally occurring deoxynucleotides may be used to enhance the extension reaction by promoting strand slippage. Numerous unnatural triphosphates have been shown to be incorporated by polymerases and many introduce a group during template dependent chain extension that has weaker binding than the base that would naturally be incorporated. Examples of unnatural triphosphates include inosine triphosphate, dUTP, 5-Br-dUTP, 5-Br-dCTP, 7-deaza-dGTP, 8-Br-ATP, 5-aminoallyl-UTP, and the like. See for example, Kool ET, “Hydrogen bonding, base stacking, and steric effects in DNA replication.”, Annu Rev Biophys Biomol Struct 2001;30:1-22; Moran S, Ren RXF, and Kool ET, “A thymidine triphosphate shape analog lacking Watson-Crick pairing ability is replicated with high sequence selectivity.”, Proc. Natl. Acad. Sci. USA 1997; 94: 10506-10511. Minimally all of the NTPs or their analogs necessary to extend the extendable oligonucleotide sequence along the tandem repeat must be used.

For the template member, the 3′ terminus may be any convenient entity, being extendable or not, where extension will serve to enhance the stability of the hybridized complex. While not required, the 5′ end of the tandem repeat need not be at the 5′ end of the template member, which may have any number of bases provided that NTPs complementary to these bases are not used in the extension reaction. Alternately the 5′ end may be attached to a nonpolynucleotide chain so long as it does not interfere with the extension as described above.

In performing the repetitive extension, the different components of the reaction mixture may be combined simultaneously or successively, complexed or uncomplexed, preferably successively. How the different components are combined will depend on the nature of the sample, the nature of the components and intended application. In addition to the two components, the reaction mixture will comprise the sample and a polymerase and at least one NTP. Frequently it will be desirable to add at least one of the polymerase and NTPs after the other components have been combined. Where it is necessary for the 3′ end of the extendable oligonucleotide component to be extended in order to form a duplex with the tandem repeat or it is desired to extend the 3′ end of the template member, it may be necessary to have more NTPs than required for replication. Extension of the 3′ end of the template member provides for greater stability of binding between the template member and the extendable oligonucleotide or other nucleic acid to which the template member is attached. However extension of the 3′ end may retard slippage and repetitive extension of the extendable oligonucleotide. In this circumstance increased repetitive extension may be achieved by including as an additional component an oligonucleotide sequence that does not have a sequence that can bind to the extendable oligonucleotide other than the tandem repeat sequence and is incapable of being extended along the extendable oligonucleotide.

To enhance slippage the 3′ end of the template repeat of the template member or any additional component with a template repeat will often be designed to have a low binding affinity to the extendable oligonucleotide component. This can be achieved by introducing a base mismatch or an unnatural base such as one or more phosphorothioates near the 3′ end of the tandem repeat. In some cases where the template tandem repeat is at the 3′ end of the template member it may be desirable to block the 3′ end to prevent extension along the extendable oligonucleotide component using a non-extendable terminal member, e.g. unnatural nucleotide, other than a nucleotide, a blocked nucleotide such as a dideoxyribonucleotide, a mismatched nucleotide, the absence of the NTP necessary for extension, or the like. Alternatively the extendable oligonucleotide component may have at its 3′ end a sequence of bases complementary with the tandem repeat sequence, containing one or more mismatches or unnatural bases such as abasic deoxyribophosphate, or mismatching nucleotides.

The method is isothermal, so that the temperature will be in the range of about 10 to 80° C., where the temperature is selected in relation to the melting temperature of the double stranded nucleic acid, enhancement of slippage, rate of extension, and the like. The medium will be a buffered salt medium in accordance with the nature of the polymerase and the binding of the various nucleic acids involved with the determination. Various polymerases may be used, such as the DNA polymerases: E. coli DNA polymerase and its Klenow fragment, modified T7 DNA polymerase, human DNA polymerase, etc., particularly exonuclease deficient polymerases. Each of the polymerases will have a preferred medium for enhanced processivity in accordance with the supplier of the enzyme.

In carrying out the present method, an aqueous medium is employed. Other polar cosolvents may also be employed, usually oxygenated organic solvents of from 1-6, more usually from 1-4, carbon atoms, including dimethylsulfoxide, alcohols, ethers, formamide and the like. Usually these cosolvents, if used, are present in less than about 70 weight percent, more usually in less than about 30 weight percent.

The pH for the medium is usually in the range of about 4.5 to 9.5, more usually in the range of about 5.5-8.5, and preferably in the range of about 6-8. Various buffers may be used to achieve the desired pH and maintain the pH during the determination. Illustrative buffers include borate, phosphate, carbonate, Tris, barbital and the like. A metal ion such as magnesium ion is usually present in the above medium.

The concentrations of the reagent components, the other reagents and the sample will vary over a wide range, depending upon the nature of the sample and the components. The ratio of the two reagent components will vary widely depending upon whether there is a separation step prior to the addition of the template component to the combined sample and extendable component. Desirably, the template member will be in excess to the extendable oligonucleotide component. The amount of the two reagents will be different when there is a separation step, since the effective concentration of the component that becomes bound will usually be substantially lower than the amount initially added. The amount of the template member that is added may be much smaller than the amount of the extendable oligonucleotide component that was originally added, since only a small proportion of the extendable oligonucleotide component will be present. Where there is no separation step, generally the reagent components will be in a molar range of about 10⁻¹⁰-10⁻⁴ M, more usually 10⁻⁸-10⁻⁶ M, depending on the binding affinity, desired rate of binding, etc., where the template member will usually be in excess, generally in the range of about 2 to 10⁴-fold excess or greater.

As one illustration of the subject invention, one of the reagent components will have a nucleic acid hybridizing region complementary with the target sequence. The target sequence may be present in a chromosome or fragment of a chromosome, cDNA, mRNA, synthetic nucleic acid, natural or unnatural, or the like. The extendable oligonucleotide component may bind directly to the target sequence, so as to hybridize to the target sequence. By having an additional region at the 3′ end of the extendable oligonucleotide that will hybridize to the template member, one can provide for amplification of signal. In this situation, there will usually be a separation step to remove the extendable oligonucleotide component that is not specifically bound to the target. The recognition region between the two components may be the tandem repeat sequence or a different sequence and will usually be shorter than the entire tandem member.

Of particular interest is the use of a stem/loop nucleic acid target detection agent for the determination of the presence of a nucleic acid sequence. One approach is described in application Ser. No. 09/805,674, filed Mar. 13, 2001, and provisional application No. 60/312,505, filed Aug. 13, 2001, which are incorporated herein by reference. Another approach is described in U.S. Pat. No. 5,925,517. These applications describe methods and compositions for identifying at least one nucleic acid sequence in a complex nucleic acid sample employing a probe, which has two complementary sequences, which are hybridized to each other (the stem) and connected by a linker. Upon binding of these probes to a complementary target polynucleotide sequence, hybridization of the complementary sequences is disrupted. The structure of these probes may be referred to as a stem and loop (stem/loop) or hairpin. Stem/loop probes that are useful in the present invention have (1) a first oligonucleotide sequence, (2) a second oligonucleotide sequence terminating in an extendable 3′ end that is complementary to and hybridized with at least a portion of the first oligonucleotide sequence thereby creating a hybridized region, (3) a linker connecting the first and second oligonucleotide sequences, and (4) a target recognition region which is complementary to the target polynucleotide and contiguous with the first oligonucleotide sequence. Depending on the probe that is used the target recognition region may be comprised of the linker alone or the first oligonucleotide sequence in combination with either the linker or a sequence at the 5′-end of the first oligonucleotide sequence. Binding of any of these probes to target leads to dissociation of the duplex formed between the first and second oligonucleotide sequences and formation of a single stranded second oligonucleotide sequence having a free and extendable 3′ end. The hybridizing conditions are selected so that dissociation of the hybridized regions occurs almost solely as a result of binding by target. Release of the second oligonucleotide sequence is detected as indicative of the presence of the target sequence present in the sample. The second oligonucleotide sequence is used as the extendable oligonucleotide component of the reagent and the template member binds to the available second oligonucleotide sequence for initiation of the extension.

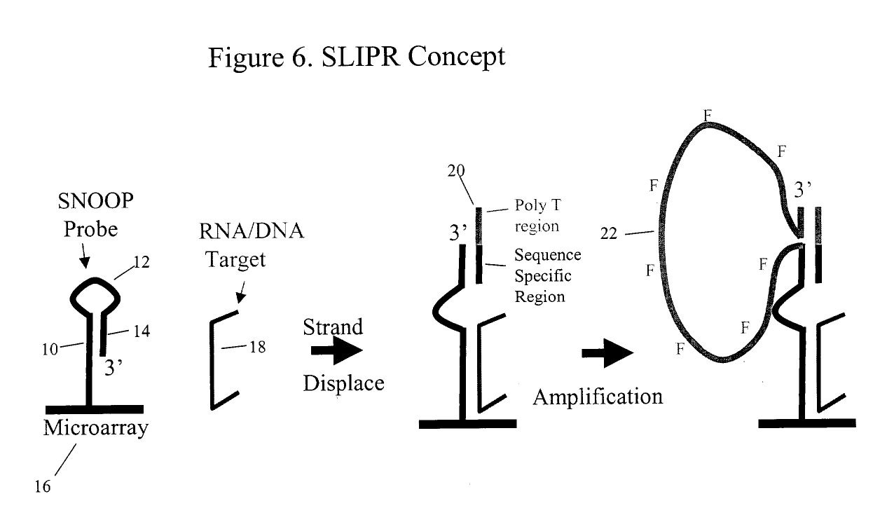

The stem/loop probes that are used in this invention are depicted in FIG. 6. The stem/loop probe 10 has a first oligonucleotide sequence 11, a linker 12 and a second oligonucleotide sequence 14 hybridized to the first oligonucleotide sequence 11 to form a double stranded stem. The first oligonucleotide sequence 11 is linked, optionally through an attachment sequence 15, to a surface 16 referred to as a “Microarray,” intending that there be a plurality of different stem/loop probes to identify a number of different targets in a nucleic acid mixture. An RNA and/or DNA mixture is added to the Microarray, where a complementary target strand 18 binds to the probe 10 at the loop 12 or along the first oligonucleotide sequence 11 and the attachment sequence 15 to release the second oligonucleotide sequence 14 with formation of the target-probe complex, 17. This makes the second oligonucleotide sequence 14 available for hybridizing to the template member 20, having a recognition region 21 for binding to the second oligonucleotide sequence 14 and a template tandem repeat, where in this illustration the tandem repeat is the polyT region. In the presence of a DNA polymerase and dATP, the 3′ terminus of the second oligonucleotide sequence 14 is repetitively extended along the polyT region to form an extended sequence 22. Additionally the 3′ end of the template member is extended along the stem/loop probe with displacement of the bound target to provide a fully double stranded complex from which sequence 22 extends. By using fluorescent labeled dATPs, the extended sequence 22 will have a plurality of fluorescers 24 indicated as “F” bound to it. By washing away the fluorescent dATPs or using a method for detecting surface fluorescence in the presence of solution fluorescence such as a method based on total internal reflection, one can detect the fluorescers bound to the surface indicating the presence of the target sequence complementary to the stem/loop probe.

The design of the stem/loop probes depends on where the probe binds to the target polynucleotide relative to suspected sequence differences in the samples. In general, the length of the hybridized portion and the single stranded region of the probes of the invention depend on the hybridization conditions that are to be used. For example, long single stranded regions are required to permit hybridization when higher temperatures are needed to avoid interference due to formation of secondary structures of a target polynucleotide. When it is desired to avoid spontaneous dissociation of the strands at higher temperatures, longer double stranded portions of the probe will be required. The first oligonucleotide sequence generally has a hybridized region of about 5 or more, usually about 8 or more nucleotides, and usually less than about 35, more usually, less than about 20 nucleotides, that are complementary to the second sequence. While longer sequences may be employed they are disadvantageous in requiring the synthesis of larger molecules. There is no critical upper limit to the number of nucleotides in the hybridized region other than any practical problems associated with preparing very long probes. The length of the single stranded region of the first oligonucleotide sequence is usually at least about 6 nucleotides and may be at least about 15 or more nucleotides, generally being not more than about 30. The subject invention provides high specificity for the polynucleotide sequence with a probe that is usually fewer than 80 bases, conveniently 35 nucleotides or fewer, generally not fewer than 17 nucleotides, excluding any nucleotides in the linker or loop. Practical considerations will generally have the single stranded tail portion of the hairpin in the range of about 11 to 23 nucleotides, the stem will generally be in the range of about 6-20 nucleotides, and the loop, when it is an oligonucleotide will generally be in the range of about 3 to 30 nucleotides.

Short single stranded regions, preferably fewer than about 20 nucleotides, will be preferred when mismatches are suspected in the portion of the target complementary to the single stranded region. When mismatches are suspected in the portion of the target polynucleotide complementary with the hybridized region, there is no critical upper limit to the number of nucleotides in either the single stranded region or double stranded stem other than matters of practicality.

The second oligonucleotide sequence of the stem/loop probes is usually identical in length and has a sequence terminating at its 3′ end that is complementary with the hybridized region of the first oligonucleotide sequence. However it is often desirable to prevent the 3′ end of the second oligonucleotide sequence from extending along the first oligonucleotide sequence when the sample is combined with the stem/loop probe in the presence of a polymerase and nucleotide triphosphates. Blocking is best achieved by having at the 3′ end of the second oligonucleotide sequence one or more bases, conveniently not more than about 3, that do not hybridize to the first oligonucleotide sequence. The additional bases that are used should be hybridizable with the template member in order to permit extension along the tandem repeat, but may include a mismatch or abasic entity.

The linker is a group involved in the irreversible attachment or binding or linkage of the first and second oligonucleotide sequences. The linkage may be covalent or non-covalent. When the linkage is non-covalent the linker will usually comprise a duplex of two complementary nucleic acid strands, each covalently attached to one of the oligonucleotide sequences. The duplex comprises sequences that do not dissociate during the use of the probe in the present method. This may be accomplished by constructing a duplex that is long enough to avoid melting under the intended assay conditions. Preferably, the duplex has a relatively high G/C content or is double stranded RNA or is comprised of PNA.

When the linker is covalent, it may be a bond but is usually a group that is polymeric or monomeric and comprises a bifunctional group convenient for linking the two sequences. Polymeric linkers may comprise, for example, an oligonucleotide or related polyalkenylphosphate, a polypeptide, a polyalkylene glycol, e.g. polyethylene glycol, and the like. Monomeric linkers may comprise, for example, alkylenes, ethers, amides, thioethers, esters, ketones, amines, phosphonates, sulfonamides, and the like. The linker may be hydrophilic or hydrophobic, preferably hydrophilic, charged or uncharged, preferably uncharged, particularly cationically charged, and may be comprised of carbon atoms and heteroatoms, such as oxygen, nitrogen, phosphorous, sulfur, etc. In this invention, the linker need not be an oligonucleotide, although oligonucleotides may be used, where the sequence may be designed for sequestering the probe, binding of a labeled complementary sequence, or other means of identification. Alternatively, the sequence when other than an oligonucleotide, may be aliphatic, alicyclic, aromatic, heterocyclic, or combinations thereof, particularly aliphatic, being a chain of from about 5 to 25 atoms, allowing flexibility in the probe, and keeping the two polynucleotide strands together.

The two ends of the linker are attached covalently to the first and second oligonucleotide sequences, respectively, in a manner that does not interfere with hybridization capabilities of the two sequences. Thus, the linker may be linked to any nucleotide or a terminus of each oligonucleotide sequence. When attachment is to a non-terminal nucleotide, it frequently is at the 5-position of U or T, the 8-position of G, the 6-amino group of A, a phosphorus atom, or the 2′-position of a ribose ring. Usually, it is most convenient to attach the linker to one of the termini of each oligonucleotide sequence. Attachment to the 5′ terminus of each sequence will often be convenient when the linker is not an oligonucleotide. Attachment at the opposite termini, that is, the 3′-end of the first oligonucleotide sequence and the 5′-end of the second oligonucleotide sequence, is convenient when the linker is an oligonucleotide or polyalkenylphosphate.

Common functionalities that may be used in forming a covalent bond between the linker and the nucleotide of the sequences to be conjugated are alkylamine, amidine, thioamide, ether, urea, thiourea, guanidine, azo, thioether and carboxylate, sulfonate, and phosphate esters, amides and thioesters. Various methods for linking molecules are well known in the art; see, for example, Cuatrecasas, J. Biol. Chem. (1970) 245:3059.

As described in these applications, a target polynucleotide is contacted with a stem/loop probe causing binding of the target with release of the second oligonucleotide sequence as a single strand that terminates in a 3′-end. Present in the reaction mixture or added subsequently are a template member, a template dependent DNA polymerase and at least one, there may be two or more NTPs, and optionally an oligonucleotide sequence similar to the template member. The oligonucleotide will not have a sequence that can bind to the extendable oligonucleotide other than the tandem repeat sequence and is incapable of being extended along the extendable oligonucleotide. The template member has a tandem repeat comprised of at least about 12 bases that is 5′ of at least a 6-base sequence complementary to the 3′-end of the second oligonucleotide sequence of the stem/loop probes. The tandem repeat and complementary sequence of the template member are linked by an oligonucleotide sequence of arbitrary length, conveniently 0 to 30 bases. The linking sequence will in some instances be comprised of modified bases such as phosphorothioates designed to weaken binding to its complementary sequence, but allowing for replication of the complementary strand. When target is present, the second oligonucleotide sequence of the stem/loop probe will bind to the complementary sequence of the template member and be extended along the template tandem repeat by means of the polymerase. Because of strand slipping this extension will be very much longer than the template tandem repeat. Minimally the NTPs must include the bases that are complementary to the tandem repeat. Frequently all four dNTPs will be present.

Extension can in principle proceed indefinitely thus providing high levels of signal amplification. Detection of the extended stem/loop probe is achieved by including a labeled NTP in the reaction mixture, which becomes incorporated into the extended probe. Alternatively, the template member can be labeled. As the extension occurs many copies of the template member become bound to the extension, thus providing for an amplified signal

For multiplexed analysis of multiple targets, the relevant stem/loop probes will be bound to different sites on a surface, where the surface may comprise an addressable array or addressable particles. Because the 3′-end of the probe is capable of being extended, attachment to the surface is at another position, conveniently the 5′-end of the probe.

An important advantage of the method is that it permits both signal amplification and selective incorporation of different types of labels. This is particularly useful when it is desired to compare a signal at a site on an array with a reference signal at the same site in order to make quantitative determinations. For this purpose two or more different template members will be used that have sequences complementary to different oligonucleotides bound to the same site. Each template member will have a different tandem repeat and be labeled with a different detectable label. Alternatively when each of the tandem repeats has at least one base that is not shared with the other tandem repeat the NTPs complementary to the unique bases can be labeled with different detectable labels. For example one tandem repeat could be (AAT)n and the other (AAAC)n. dATP, dTTP and dGTP would then be required for extension, and dATP and dGTP would be at least partially labeled with different fluorescent labels. Other examples of acceptable pairs of tandem repeats include (AAC)ₙ+(AAG)ₙ, (TTC)ₙ+(TTG)ₙ, (TTC)ₙ+(TATTG)ₙ, and (AAATC)ₙ+(AAATG)ₙ. For this application it will be preferable to avoid having tandem repeats that are complementary to each other so that combinations such as (A)ₙ, (T)ₙ and (AAT)ₙ, (ATT)ₙ should not be used. It is apparent from the above that a combination of one labeled template oligonucleotide and a labeled NTP that is complementary only to bases in the other template oligonucleotide would also provide dual labeling.

Similarly, slipping induced extension can be employed for detection of oligonucleotide binding to other than stem/loop probes. Thus an oligonucleotide probe can be designed such that it has a portion that hybridizes to a surface bound target polynucleotide and a sequence at its 3′-end that does not hybridize with the target. An above described template member having a tandem repeat can then be added together with a polymerase and NTPs to cause extension and labeling of the bound probe. This approach is particularly useful for sandwich assays in which targets are bound to an array of capture probes or addressable beads. A mixture of target polynucleotides and the corresponding oligonucleotide probes can be combined with the array causing binding of the probes at sites to which target becomes bound. The array is then contacted with the template oligonucleotide and other reagents and the detection of label at specific sites on the array provides an indication of the presence of a specific target.

Still another application of the method is for painting of chromosomes (FISH). In this application a probe having a region complementary to a sequence in the chromosome is used that has a 3′-end that does not hybridize to the chromosome. After binding is complete the template member is added together with a polymerase and NTPs, at least one of which is labeled with a fluorescent dye. This procedure is preferable over the use of a probe that has a long labeled sequence preattached because it avoids steric hindrance to binding associated with the large probes.

The method also has application in homogeneous binding assays. For example, two probes can be used that bind to different sites on a target polynucleotide. One of the probes will carry a first label. The other probe will be designed to bind to the target while leaving an unhybridized 3′-end comprised of at least 6 bases. A template member as described above, polymerase, and NTPs including a second label are then added to initiate chain extension of the probe and incorporation of multiple copies of the second label. Thus only when the target is present will the first label be incorporated into the polymer comprised of many second labels. The means of detection will depend on the pair of labels that is used. For example the first label can be a fluorescent energy acceptor and the second label a chemiluminescent compound such as an acridinium ester. Upon activation of the acridinium ester with base, emission from the first label will be observable only when target is present.

An important extension of the homogenous method is for haplotyping and detection of splice variants. For this purpose one set of stem/loop probes is assembled in an array and a second set of stem/loop probes is provided along with target DNA or RNA for target dependent binding to the array. Slipping induced extension of the two released stem/loop probe 3′-ends permits independent incorporation of two different labels as described above. The labels can comprise a pair of different fluorophores, a fluorophore and a chemiluminescent compound, a pair of different enzymes, etc. Detection of both labels at the same site indicates the presence of sequences complementary to two probes in a single target molecule. Detection of only one label indicates the presence of one of the complementary sequences in the target.

The subject reagent may be used as a label in almost any type of assay, where a ligand and receptor are involved. For this purpose, an oligonucleotide serving as a recognition sequence and having an extendable terminus may be employed. Conditions must be employed that allow for distinguishing between the presence and absence of a complex between the ligand and receptor. For this purpose, one may use bound ligand or receptor, where the complex member may be initially bound to a surface or becomes bound after the sample and the reciprocal binding member have been brought together under complex formation conditions. Numerous “heterogeneous” assays are known and commercially available, such as ELISA, RIA, fluorescent assays, etc. In these assays there is a label that is detectable, that may be replaced by the extendable oligonucleotide component. By bonding the extendable oligonucleotide component to one of the complex members and providing for binding of the labeled member to a surface as a function of the presence of an analyte, one obtains an amount of label bound to the surface related to the amount of complex bound to the surface. In the present method, one would then add the template member, polymerase and one or more dNTPs to initiate the repetitive extension. As discussed previously, by having a labeled dNTP that becomes incorporated into the amplified sequence, the amount of label may be determined and related to the amount of complex formed.

One may use either a competitive or a sandwich format, where an analyte-binding agent is on a surface and a second labeled reagent is used. The second labeled reagent may bind respectively either to the binding agent in competition with the analyte or bind to analyte that is bound to the binding agent. In either event the second binding member is labeled with the extendable oligonucleotide component, which, as previously described, is extended by means of the template member and NTPs, at least one of which carries a detectable label.

The subject reagent may be used with receptors associated with cell membranes, where the number of receptors is low and amplification is required in order to detect the receptors. The method would follow the same procedure as if the receptors were bound to a solid surface, where the cell membrane provides the surface. The cells may be dispersed and afterwards concentrated using centrifugation, filtration, etc. The signal may then be read free of interference from label in solution.

For convenience, kits can be provided comprising the various reagents necessary for performing the method including a reagent comprised of two oligonucleotide components comprising a tandem repetitive sequence as a template and a 3′ extendable terminus For determining target nucleic acids, one of the oligonucleotide components will have a recognition region capable of binding directly to the target nucleic acid or to a probe that is capable of binding to the target nucleic acid. One may have a plurality of oligonucleotide components with different recognition regions related to the different target nucleic acids. In addition, one may have one or more probes including stem/loop probes, each probe related to a different target nucleic acid. In this way there are pairs of probes and oligonucleotide components for multiplexed determination of different nucleic acid targets in a mixture. Conveniently, the probes can be bound to a surface, where the position of each probe is related to the sequence of the probe. The surface can be a particle, with different particles for different sequences, a flat surface to provide an array, etc. For binding events where one of the reagents is not a nucleic acid, such as ligands and receptors, usually involving a protein, one can have one of the binding members labeled with an oligonucleotide, usually having an extendable 3′ terminus, although the oligonucleotide can be the tandem repeat. Depending on the choice of oligonucleotide, the nucleic acid binding to the oligonucleotide label will have a tandem repeat or a 3′ extendable terminus. Where it is desired that the nucleic acid serving as the tandem repeat not be extended, then extension can be blocked in a variety of ways, including an unnatural terminal nucleotide including a dideoxy terminal nucleotide, a blocked terminal nucleotide, a terminal member other than a nucleotide or the absence of NTPs for extension.

In addition to the specific reagents, kits may include a DNA polymerase and NTPs, particularly including labeled NTPs, more particularly fluorescer labeled NTPs.

The following examples are intended to illustrate but not limit the invention.

### EXPERIMENTAL

The following are detailed descriptions of the figures relating to the experimentation associated with demonstrating the subject invention.

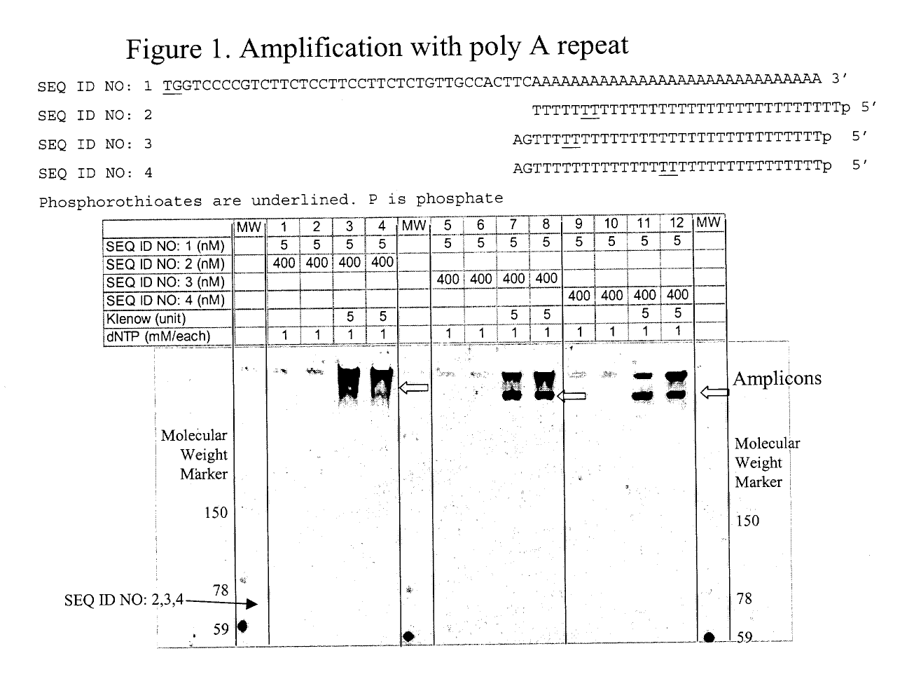

FIG. 1, Repetitive extension with poly A repeat.

This figure illustrates the repetitive extension. The four DNA oligos are listed on the top. SEQ ID NO: 1 oligo contains poly A repeats, and the complementary oligos (SEQ ID NOS: 2, 3, and 4) contain poly T repeats. The gel shows the Klenow extension resulting when mixing the four oligos in different combinations. In the presence of dNTPs and Klenow DNA polymerase, when SEQ ID NO: 1 was combined with any one of the other three sequences (SEQ ID NOS: 2, 3, and 4 ), DNA amplicons (the amplified sequence resulting from replication of the complementary sequence) showed up with large molecular weights (lanes 3, 4, 7, 8, 11, 12). High molecular weight products were observed only in the presence of the polymerase. T (lanes 1, 2, 5, 6, 9, and 10).

FIG. 2, Mechanism of the Repetitive Extension with Poly A Repeats.

The proposed repetitive extension mechanism is shown when SEQ ID NO: 1 and SEQ ID NO: 4 are mixed for Klenow polymerase extension (lanes 7 and 8 of the gel in FIG. 1). SEQ ID NO: 4 first hybridizes to SEQ ID NO: 1., Kienow polymerase then extends SEQ ID NO: 4 along SEQ ID NO: 1, resulting in a duplex. The poly A portion of the SEQ ID NO: 1 slides along SEQ ID NO: 4 in the 3′ to 5′ direction resulting in a bulge in the middle of the duplex, leaving the 3′ end of SEQ ID NO: 1 recessive. Klenow polymerase then extends the recessive 3′ end of SEQ ID NO: 1 along the poly T region of SEQ ID NO: 4. This sliding and Klenow polymerase extension cycle can repeat resulting in a greatly elongated SEQ ID NO: 1, the amplicon. When only dATP is present without dTTP, dGTP, or dCTP, the repetitive extension can still occur but SEQ ID NO: 4 is not extended along SEQ ID NO: 1.

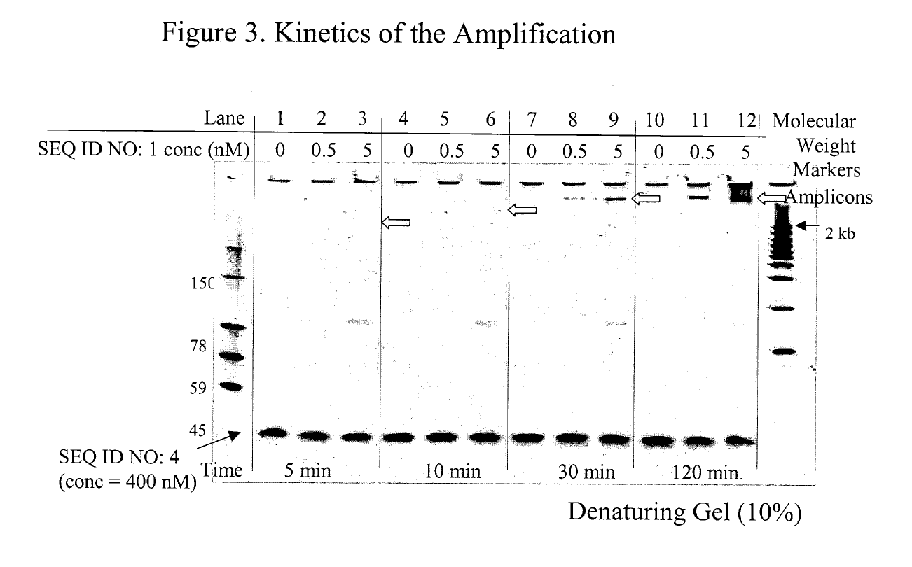

FIG. 3, Kinetics of the Repetitive Extension

To further evidence the mechanism of the repetitive extension, the repetitive extension was monitored with respect to time of incubation. The first three lanes show a titration of the SEQ ID NO: 1 concentration (0, 0.5, and 5 nM) with a fixed 400 nM concentration of SEQ ID NO: 4. All three experiments were monitored after incubation periods of 5 min, 10 min, 30 min, and 120 min. With a SEQ ID NO: 1 concentration of 5 nM (lanes 3, 6, 9, and 12), the increase in amplicon size with time is amply evidenced by progressively slower migration in the gel. The growing size of the product can also be observed at 0.5 nM of SEQ ID NO: 1 (lanes 2, 5, 8, and 11), but less amplicon is produced at each time period as illustrated by weaker intensities of the lines. When SEQ ID NO: 1 is absent (lanes 1, 4, 7, and 10), no repetitive extension is observed. This experiment confirms that the amplicons grow with time; there is a dose response with SEQ ID NO: 1; and SEQ ID NO: 4 alone cannot lead to observable repetitive extension.

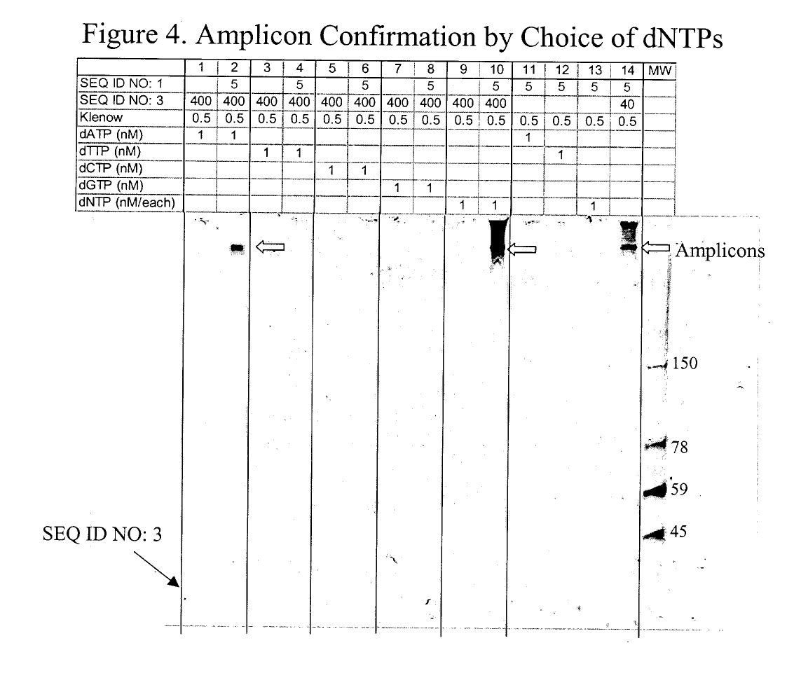

FIG. 4, Confirmation of the Repetitive Extension Process by Choice of dNTPs

To further confirm the repetitive extension mechanism, the repetitive extension was performed with different dNTP combinations. Only when dATP was present could repetitive extension occur (lanes 2, 10, and 14). No repetitive extension was observed when dATP was absent (lanes 4, 6, and 8). This indicates that dATP is the only building block needed for repetitive extension, agreeing with the process described in FIG. 2. In the absence of SEQ ID NO: 1, again no repetitive extension was observed (lanes 1 and 9). In the absence of SEQ ID NO: 3 (lanes 11, 12, and 13), there also was no repetitive extension observed. When the SEQ ID NO: 3 to SEQ ID NO: 1 ratio is dropped from 80:1 (lane 10) to 8:1 (lane 14), there is less repetitive extension.

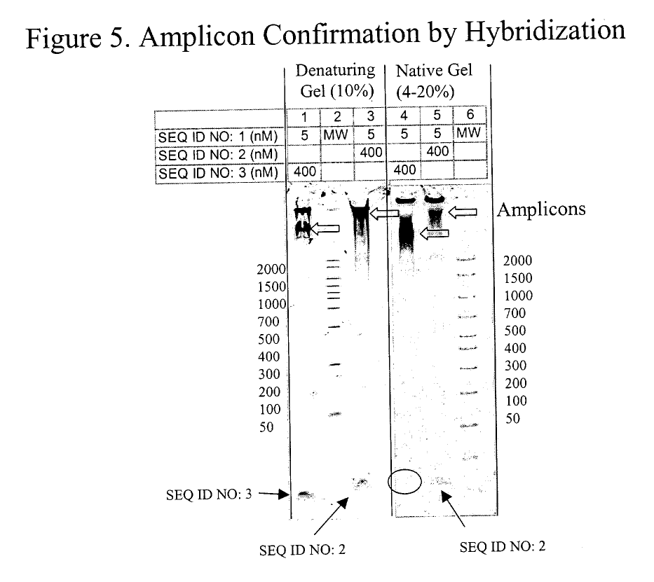

FIG. 5, Amplicon Formation Confirmation by Hybridization

To confirm hybridization between excess SEQ ID NO: 3 and the duplex formed as a result of repetitive extension with strand slippage, native gel was used, so as not to disturb any hybrids in the repetitive extension mixture. FIG. 5 shows a comparison between a denaturing gel on the left and a native gel on the right. Comparison between lanes 1 and 4 clearly showed hybridization. Excess SEQ ID NO: 3 appears in the denaturing gel (lane 1), but disappears in the native gel (lane 4), where it is believed to be hybridizing to the bulge portion of the amplicon. Since there is an 80 fold excess of SEQ ID NO: 3 relative to SEQ ID NO: 1, and SEQ ID NO: 3 has a 30 T repeat, the extended portion may be estimated to be at least 80×30=2,400 nucleotide long.

Interestingly in the repetitive extension of SEQ ID NO: 1 using SEQ ID NO: 2, not all of the excess of SEQ ID NO: 2 hybridizes to the bulge (lane 5 vs. lane 3). This may be attributed to the fact that SEQ ID NO: 2 is purely poly T, unlike SEQ ID NO: 3 which has a poly T attached to GA at the 3′ end (see FIG. 1). As a result, when it hybridizes to the bulge region, SEQ ID NO: 2 is extended along the bulge, making the bulge mostly double stranded, and not accessible by most of the excess SEQ ID NO: 2. SEQ ID NO: 3, on the other hand, cannot extend along the bulge because of the two mismatched (G/A and A/A) at the 3′ end. Therefore, there is enough binding space on the bulge for SEQ ID NO: 3 to hybridize.

FIG. 6, SLIPR Concept.

Stem/loop probe initiated polymerization or SLIPR is described as a combination between the use of a stem/loop probe and repetitive extension. A stem/loop probe is bound to the solid phase at its 5′ end. A DNA or RNA target hybridizes and strand-displaces the stem/loop probe, leaving the 3′ end available for hybridization with a poly T template member with the 3′ end blocked. The 3′ end of the stem/loop probe can therefore be extended by Klenow polymerase along the template, forming a poly A/poly T duplex, which initiates the repetitive extension. Fluorescent dNTPs can be incorporated in the amplicon leading to multiple fluorophores attached to each stem/loop probe. See earlier discussion in paragraph 40.

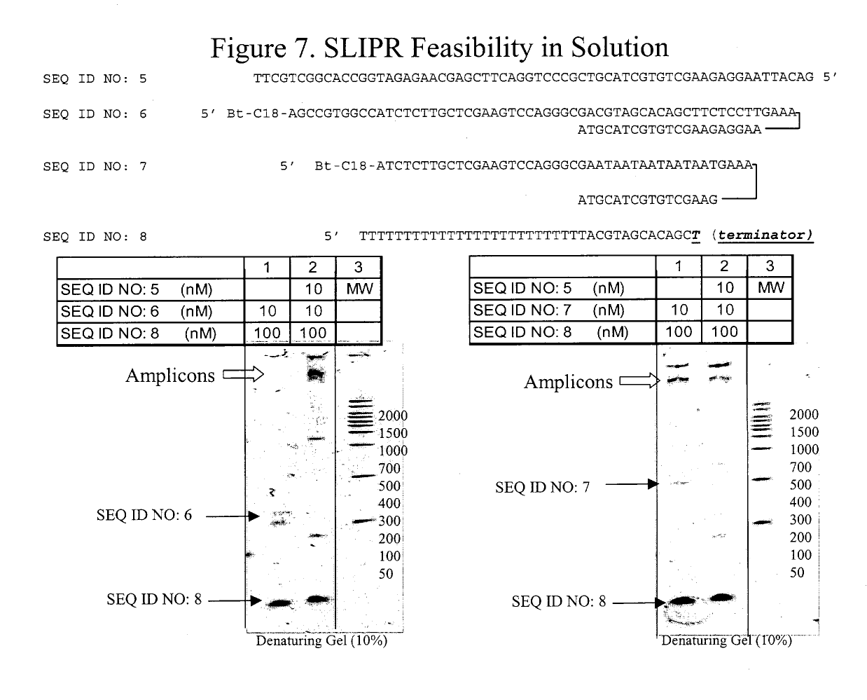

FIG. 7, SLIPR Feasibility in Solution

A synthetic DNA oligonucleotide target sequence is shown as SEQ ID NO: 5. A stem/loop probe (SEQ ID NO: 6) forms a 20 base pair stem/loop structure with GAAA in the loop and a single base A dangling at the 3′ end. A linear probe (SEQ ID NO: 7) is a control sequence with the 3′ end always available for hybridization. A poly T template member (SEQ ID NO: 8) has 14 bases at the 3′ end complementary to the 14 bases at the 3′ ends of the stem/loop probe (SEQ ID NO: 6) and the linear probe (SEQ ID NO: 7). SEQ ID NO: 8 has its 3′ end blocked by a dideoxy terminator, so that it is not extendable, but can serve as template.

In the left gel, the stem/loop probe (SEQ ID NO: 6) is combined with the template member (SEQ ID NO: 8). Repetitive extension only occurs when the target (SEQ ID NO: 5) is present (lane 2). The negative control is clean (lane 1). In the right gel, linear probe (SEQ ID NO: 7) is mixed with template member (SEQ ID NO: 8). Repetitive extension occurs with (lane 2) or without (lane 1) target (SEQ ID NO: 5). The use of the SLIPR protocol in solution is shown to be operative.

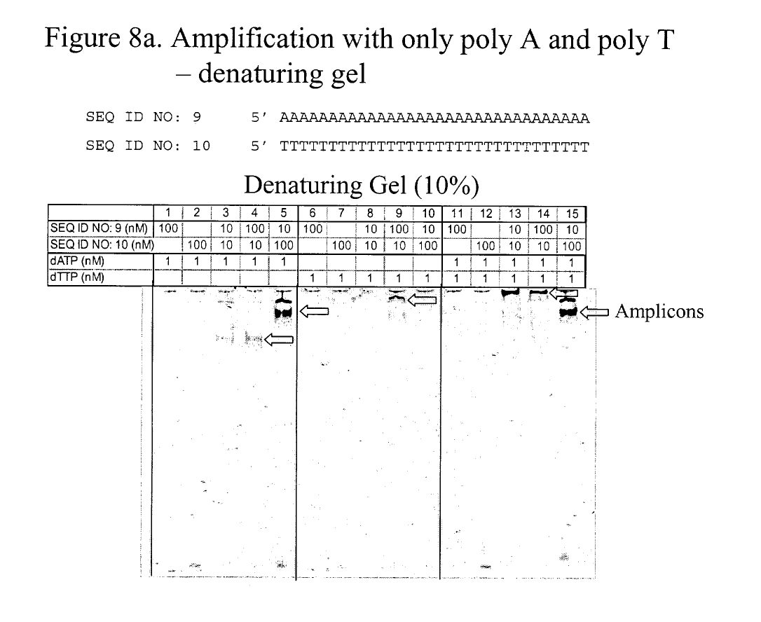

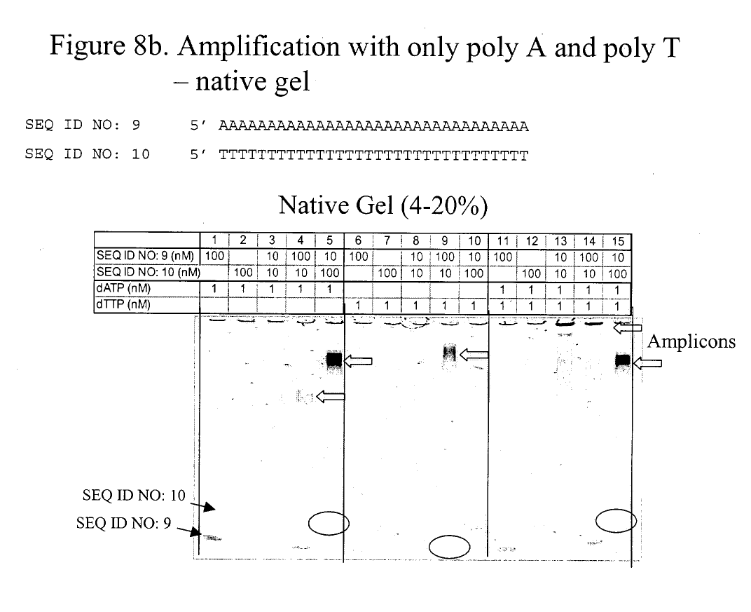

FIGS. 8a and 8b, Repetitive extension amplification with only poly A and poly T

To demonstrate the generality of the repetitive extension amplification, oligonucleotides with poly A only (SEQ ID NO: 9) and poly T only (SEQ ID NO: 10) were tested together with dATP (lanes 1-5), dTTP (lanes 6-10), or both (lanes 11-15). The same experiments were run on a denaturing gel (FIG. 8a) and a native gel (FIG. 8b). Neither poly A nor poly T can repetitively amplify by themselves (lanes 1, 2, 6, 7, 11, and 12).

When only dATP was present, only poly A can extend on the poly T template (lane a lanes 3, 4, and 5). When poly A and poly T are mixed in 1:1 ratio (lane 3), medium size amplicons appear. When the proportion of poly A is in excess (lane 4), there is no increase in the amount of the amplicons, because the excess poly A has no template to extend on. When poly T is in excess (lane 5), a larger size amplicon appears. It is believed that the band for poly T is missing since it is believed to be hybridizing to the amplicon (lane 5, FIG. 8b).

When only dTTP is present, only poly T can extend on the poly A template (lanes 8, 9, and 10). When poly T and poly A are mixed in 1:1 ratio (lane 8), no repetitive amplification is observed. When poly A is in excess (lane 9) moderate repetitive amplification is observed. Also poly A is missing from the gel, presumably because it hybridizes to the amplicon (lane 9, FIG. 8b). When poly T is in excess (lane 10), no repetitive amplification is observed.

When both dATP and dTTP are present, both poly A and poly T can extend on each other. Poly T remains double stranded (lane 13), excess of poly A does not help (lane 14), because excess poly A has no poly T to bind. But surprisingly excess poly T does help (lane 15), probably because slow extension of poly T makes poly A single strand available for hybridization with poly T. Hybridization is confirmed by the missing poly T band (lane 15, FIG. 8b).

In repeating the experiments described in FIG. 8a, some differences in result were observed. For the protocol of lane 10, an amplicon band was observed in two other runs, and in one out of two runs an amplicon band was observed for the protocol of lane 8.

### Materials and Methods.

DNA sequences were chemically synthesized by standard phosphoramidite chemistry at Oligo Etc (Wilsonville, Oreg.). Klenow fragment, without 5′ exonuclease activity, of DNA polymerase I, and exonuclease lambda were purchased from USB Corporation, Cleveland, Ohio. Four deoxy NTPs were purchased from Roche Biosciences, Indianapolis, Ind. The reaction buffer used in all experiments is Klenow reaction buffer purchased from USB Corporation. Mg buffer contains 10 mM MgCl₂ and 20 mM Tris, pH 8. TE buffer and 500 μl reaction tubes were purchased from Ambion Inc. Austin, Tex. 10,000X SYBR Gold gel stain was purchased from Invitrogen Corporation, Carlsbad, Calif.

Twenty μl reaction protocol is as follows. Mix all the oligos with 2 μl of Mg buffer, and appropriate amount of TE buffer so that the total final volume will be 20 μl. Incubate at 95° C. for 30 s and cool down to room temperature by air for 20 min. Add mixture of 2 μl of dNPTs (10 mM of each dNTP), 2 μl of 10X Klenow buffer, 5.5 μl of H₂O, and 0.5 μl of Klenow DNA polymerase (5 unit/l). Incubate at 37° C. for 30min. Add gel-loading buffer (from Ambion), and load to 10% denaturing and 4-20% native gels. Run at 200 volts until the lower dye reaches the bottom of the gel. Stop the gel. Remove the gel from the gel cassette, and submerge into the I X SYBR gold gel stain for 30 min. Take gel from the gel staining solution and quantify the fluorescein signal on the gel and recorded gel image in the Epi Chemi II dark room (UVP Inc. Upland, Calif.). For detailed protocol, please refer to the table in each experimental figure. Nucleic Acid Sequences:

| | |
|---|---|
| TGGTCCCCGTCTTCTCCTTCCTTCTCTGTTGCCACTTCAAAAAAAAAAAAAAAAAAAAAAAAAAAAAA: | SEQ ID NO: 1 |
| (the underlined nueleotides are phosphorothioates) | |
| pTTTTTTTTTTTTTTTTTTTTTTTTTTTTTTTT | SEQ ID NO: 2 |
| pTTTTTTTTTTTTTTTTTTTTTTTTTTTTTTGA | SEQ ID NO: 3 |
| pTTTTTTTTTTTTTTTTTTTTTTTTTTTTTTGA | SEQ ID NO: 4 |
| GACATTAAGGAGAAGCTGTGCTACGTCGCCCTGGACTTCGAGCAAGAGATGGCCACGGCTGCTT | SEQ ID NO: 5 |
| Biotin-C 18- | |
| AGCCGTGGCCATCTCTTGCTCGAAGTCCAGGGCGACGTAGCACAGCTTCTCCTTGAAAAAGGAGAAGCTGTGCTACGTA | SEQ ID NO: 6 |
| Biotin-C 18- | |
| ATCTCTTGCTCGAAGTCCAGGGCGAATAATAATAATAATGAAAGAAGCTGTGCTACGTA | SEQ ID NO: 7 |
| TTTTTTTTTTTTTTTTTTTTTTTTTTACGTAGCACAGCddT | SEQ ID NO: 8 |
| AAAAAAAAAAAAAAAAAAAAAAAAAAAAAAAA | SEQ ID NO: 9 |
| TTTTTTTTTTTTTTTTTTTTTTTTTTTTTTTT | SEQ ID NO: 10 |
| Underlined are the phosphorothioate nucleotides. | |
| p stands for phosphate. | |
| C18 is hexaethyleneglycol. | |

Although the invention has been described with reference to the above examples, it will be understood that modifications and variations are encompassed within the spirit and scope of the invention. Accordingly, the invention is limited only by the following claims.

What is claimed is:

1. In a method of detecting a label, the improvement comprising:
employing as said label a reagent having a 3′ extendable terminus hybridized to a tandem repeat template in combination with a DNA polymerase and dNTPs necessary for repetitively replicating said tandem repeat.

2. In a method of detecting a binding event between first and second binding members, employing a label to determine the occurrence of the binding event, the improvement comprising:
employing as said label a reagent comprising (1) an extendable oligonucleotide with a first recognition region and (2) a template member having a second recognition region complementary to said first recognition region and a repetitive region, wherein one of said extendable oligonucleotide and said template member is bound to said first binding member,
repetitively extending said extendable oligonucleotide with a polymerase and NTPs required for said extension along said repetitive region, to result in an extended oligonucleotide, and
detecting said extended oligonucleotide.

3. A method according to claim 2, wherein said second recognition region comprises said repetitive region.

4. A method according to claim 2, wherein said extendable oligonucleotide is joined to an oligonucleotide sequence that binds to a target nucleic acid sequence.

5. A method according to claim 2, wherein one member of said reagent is bound to a surface during said extending.

6. A method according to claim 2, wherein said NTPs comprise labeled NTPs.

7. A method according to claim 6, wherein said NTP is labeled with a fluorescer.

8. A method for detecting a binding event between complementary nucleic acid sequences, said method comprising:
combining: (1) a sample suspected of containing a target nucleic acid ; (2) a stem/loop nucleic acid probe having a first oligonucleotide sequence, a second oligonucleotide sequence complementary to said first oligonucleotide sequence having an extendable 3′ terminus, a linker joining said first and second oligonucleotide sequences, and a target recognition region contiguous with said first oligonucleotide sequence, whereby when said target nucleic acid sequence binds to said target recognition region, said second oligonucleotide sequence becomes single stranded; (3) a template member comprising a recognition region complementary to said second oligonucleotide sequence and a template region comprising tandem repeating units;
combining in a hybridizing and replicating medium said stem/loop probe and a DNA polymerase; and NTPs for replicating said tandem repeating units,
whereby said target nucleic acid binds to said stem/loop probe and said template member binds to said single stranded second oligonucleotide sequence with repetitive extension of said tandem repeating units; and
detecting said repetitive extension as indicative of the presence of said target nucleic acid in said sample.

9. A method according to claim 8, wherein said tandem repeating units are polyT.

10. A method according to claim 8, wherein said stem/loop probe is bound to a surface.

11. A method according to claim 10, wherein said stem/loop probes comprise a plurality of stem/loop probes having different nucleic acid sequences for different targets and said tandem repeating units are different for different stem/loop probes.

12. A method for detecting a binding event between first and second binding members comprising a ligand and a receptor, wherein said first of said binding members is labeled with an oligonucleotide comprising an extendable 3′ terminus, said method comprising:
combining in a binding medium: (1) said second binding member; and (2) said first labeled binding member to form a complex;
adding to said complex under polymerizing conditions: (3) a template member comprising a recognition region complementary to at least a portion of said oligonucleotide and a template region comprising tandem repeating units 4) a DNA polymerase; and (5) NTPs for replicating said tandem repeating units;
whereby said template member binds to said oligonucleotide and repetitively extends along said tandem repeating units; and
detecting said repetitive extension as indicative of binding of said first and said second binding members.

13. A method according to claim 12, wherein unbound first labeled binding member is separated from first labeled binding member bound to said second binding member following formation of said complex.

14. A method according to claim 12, wherein said recognition region comprises at least a portion of said tandem repeat region of said template member.

15. A method according to claim 12, wherein one of said binding members is an antibody.

16. A method according to claim 12, wherein said tandem repeat region is polyT.

17. A method according to claim 12, wherein said NTPs comprise a labeled NTP.

18. A method according to claim 17, where said NTPs are labeled with a fluorescer.

19. A kit comprising an oligonucleotide labeled binding member labeled with (1) at least one component of a reagent, said reagent comprising an oligonucleotide with an extendable 3′ end and a template member comprising a tandem repeat region, (2) the other component of said reagent when said binding member is labeled with only one of said components, (3) a DNA polymerase, and (4) NTPs for repetitively extending said extendable 3′ end along said tandem repeat region.

20. A kit comprising an oligonucleotide labeled binding member, said oligonucleotide having an extendable 3′ end, a labeled template member comprising a tandem repeat region, a DNA polymerase, and NTPs for repetitively extending said extendable 3′ end along said tandem repeat region.

21. A kit comprising an oligonucleotide comprising a recognition region complementary to the 3′ end of a target nucleic acid and a tandem repeat region, a DNA polymerase, and NTPs, at least one of said oligonucleotide and said NTPs comprising a label for repetitively extending said 3′ end along said tandem repeat region.

22. A kit according to claim 21, further comprising a stem/loop probe having a first arm complementary to said target nucleic acid and a second arm having an extendable 3′ end and complementary to a portion of said first arm.

23. A kit according to claim 22, comprising a plurality of different of said stem/loop probes bound to a surface and a plurality of template members having different tandem repeats related to different recognition regions.
